(19) United States
(12) Patent Application Publication
(10) Pub. No.: US 2006/0276537 A1
(43) Pub. Date: Dec. 7, 2006

(54) Use of ladostigil for the treatment of multiple sclerosis

(76) Inventors: Tamar Goren, Rehovot (IL); Eran Blaugrund, Rehovot (IL)

Correspondence Address: COOPER & DUNHAM, LLP, 1185 AVENUE OF THE AMERICAS, NEW YORK, NY, 10036, US

(21) Appl. No.: 11/443,880

(22) Filed: May 31, 2006

Related U.S. Application Data
(60) Provisional application No. 60/700,850, filed on Jul. 19, 2005.
(60) Provisional application No. 60/686,791, filed on Jun. 1, 2005.

(51) Int. Cl. A61K 31/325

(52) U.S. Cl. 514/485

(57) ABSTRACT

Disclosed are methods for the treatment of a form of multiple sclerosis comprising administering an amount of R(+)-6-(N-methyl, N-ethyl-carbamoyloxy)-N′-propargyl-1-aminoindan or a pharmaceutically acceptable salt thereof.

This application claims the benefit of U.S. Provisional Application No. 60/700,850, filed Jul. 19, 2005 and U.S. Provisional Application No. 60/686,791, filed Jun. 1, 2005, the entire contents of which are hereby incorporated by reference.

Throughout this application various publications, published patent applications, and published patents are referenced. The disclosures of these publications in their entireties are hereby incorporated by reference into this application in order to more fully describe the state of the art to which this invention pertains.

### BACKGROUND OF THE INVENTION

One of the more common neurologic diseases in human adults is multiple sclerosis. This condition is a chronic, inflammatory CNS disease characterized pathologically by demyelination in the brain and spinal cord. There are five main forms of multiple sclerosis: 1) benign multiple sclerosis; 2) relapsing-remitting multiple sclerosis (RR-MS); 3) secondary progressive multiple sclerosis (SP-MS); 4) primary progressive multiple sclerosis (PP-MS); and 5) progressive-relapsing multiple sclerosis (PR-MS). Benign multiple sclerosis is characterized by 1-2 exacerbations with complete recovery, no lasting disability and no disease progression for 10-15 years after the initial onset. Benign multiple sclerosis may, however, progress into other forms of multiple sclerosis. Patients suffering from RR-MS experience sporadic exacerbations or relapses, as well as periods of remission. Lesions and evidence of axonal loss may or may not be visible on MRI for patients with RR-MS. SP-MS may evolve from RR-MS. Patients afflicted with SP-MS have relapses, a diminishing degree of recovery during remissions, less frequent remissions and more pronounced neurological deficits than RR-MS patients. Enlarged ventricles, which are markers for atrophy of the corpus callosum, midline center and spinal cord, are visible on MRI of patients with SP-MS. PP-MS is characterized by a steady progression of increasing neurological deficits without distinct attacks or remissions. Cerebral lesions, diffuse spinal cord damage and evidence of axonal loss are evident on the MRI of patients with PP-MS. PR-MS has periods of acute exacerbations while proceeding along a course of increasing neurological deficits without remissions. Lesions are evident on MRI of patients suffering from PR-MS (Multiple sclerosis: its diagnosis, symptoms, types and stages, 2003 <http://www.albany.net/-tjc/multiple-sclerosis.html>).

The cause of multiple sclerosis is unknown. (The Merck Manual, 17th Ed., 1999, pp. 1474) Researchers have hypothesized that multiple sclerosis is an autoimmune disease (Compston, Genetic susceptibility to multiple sclerosis, in McAlpine's Multiple Sclerosis, Matthews, B. ed., London: Churchill Livingstone, 1991, 301-319; Hafler and Weiner, M S: A CNS and systemic autoimmune disease, Immunol. Today, 1989, 10:104-107; Olsson, Immunology of multiple sclerosis, Curr. Opin. Neurol. Neurosurg., 1992, 5:195-202). An autoimmune hypothesis is supported by the experimental allergic encephalomyelitis (EAE) model of multiple sclerosis, where the injection of certain myelin components into genetically susceptible animals leads to T cell-mediated CNS demyelination (Parkman, Graft-versus-host Disease, Ann. Rev. Med., 1991, 42: 189-197). Another theory regarding the pathogenesis of multiple sclerosis is that a virus, bacteria or other agent, precipitates an inflammatory response in the CNS, which leads to either direct or indirect (“bystander”) myelin destruction, potentially with an induced autoimmune component (Lampert, Autoimmune and virus-induced demyelinating diseases. A review, Am. J. Path., 1978, 91:176-208; Martyn, The epidemiology of multiple sclerosis, in McAlpine's Multiple Sclerosis, Matthews, B., ed., London: Churchil Livingstone, 1991, 3-40). Another experimental model of multiple sclerosis, Theiler's murine encephalomyelitis virus (TMEV) (Dal Canto, M. C., and H. L. Lipton. 1977. Multiple sclerosis. Animal model: Theiler's virus infection in mice. Am. J. Path. 88:497-500; Rodriguez, M. et al. 1987. Theiler's murine encephalomyelitis: a model of demyelination and persistence of virus. Crit. Rev. Immunol., 7:325), supports the theory that a foreign agent initiates multiple sclerosis. In the TMEV model, injection of the virus results in spinal cord demyelination.

Current disease-modifying drugs include interferons, glatiramer acetate (“GA”), mitoxantrone and natalizumab. (nationalmssociety.org/treatments.asp) There are currently six disease-modifying medications approved for use in relapsing forms of multiple sclerosis by the US Food and Drug Administration (FDA). Id. Four of the five medications are self-injectable drugs; one is delivered by IV infusion in a medical setting.

### SUMMARY OF THE INVENTION

The subject invention provides another treatment for multiple sclerosis comprising administration of ladostigil.

The subject invention also provides a method of treating a subject afflicted with a form of multiple sclerosis comprising administering to the subject an amount of R(+)-6-(N-methyl, N-ethyl-carbamoyloxy)-N′-propargyl-1-aminoindan or a pharmaceutically acceptable salt thereof.

The subject invention also provides a method for alleviating a symptom of multiple sclerosis in a subject afflicted with a form of multiple sclerosis comprising administering to the subject an amount of a compound effective to cause at least 60% inhibition of acetylcholinesterase in the blood of the subject thereby alleviating the symptom of multiple sclerosis in the subject.

The subject invention also provides a method for alleviating a symptom of multiple sclerosis in a subject afflicted with a form of multiple sclerosis comprising administering to the subject an amount of a compound effective to cause at least 35% inhibition of butyrylcholinesterase in the blood of the subject thereby alleviating the symptom of multiple sclerosis in the subject.

The subject invention also provides a method for alleviating a symptom of multiple sclerosis in a subject afflicted with a form of multiple sclerosis comprising administering to the subject an amount of a compound effective to cause at least 55% inhibition of monoamine oxidase A in the subject thereby alleviating the symptom of multiple sclerosis in the subject.

The subject invention also provides a method for alleviating a symptom of multiple sclerosis in a subject afflicted with a form of multiple sclerosis comprising administering to the subject an amount of a compound effective to cause at least 81% inhibition of monoamine oxidase B in the subject thereby alleviating the symptom of multiple sclerosis in the subject.

The subject invention also provides a use of R(+)-6-(N-methyl, N-ethyl-carbamoyloxy)-N′-propargyl-1-aminoindan or a pharmaceutically acceptable salt thereof in the manufacture of a medicament for the treatment of, or alleviation of symptoms of, a form of multiple sclerosis.

The subject invention also provides a use of a compound effective to cause at least 60% inhibition of acetylcholinesterase in the blood of the subject in the manufacture of a medicament for alleviating a symptom of multiple sclerosis.

The subject invention also provides a use of a compound effective to cause at least 35% inhibition of butyrylcholinesterase in the blood of the subject in the manufacture of a medicament for alleviating a symptom of multiple sclerosis.

The subject invention also provides a use of a compound effective to cause at least 55% inhibition of monoamine oxidase A in the blood of the subject in the manufacture of a medicament for alleviating a symptom of multiple sclerosis.

The subject invention also provides a use of a compound effective to cause at least 81% inhibition of monoamine oxidase B in the blood of the subject in the manufacture of a medicament for alleviating a symptom of multiple sclerosis.

The subject invention also provides a pharmaceutical composition for use in the treatment of, or alleviation of symptoms of, a form of multiple sclerosis, which comprises an amount of R(+)-6-(N-methyl, N-ethyl-carbamoyloxy)-N′-propargyl-1-aminoindan or a pharmaceutically acceptable salt thereof and a pharmaceutically acceptable carrier.

### DETAILED DESCRIPTION OF THE INVENTION

The subject invention provides a method of treating a subject afflicted with a form of multiple sclerosis comprising administering to the subject an amount of R(+)-6-(N-methyl, N-ethyl-carbamoyloxy)-N′-propargyl-1-aminoindan or a pharmaceutically acceptable salt thereof.

In a further embodiment, the form of multiple sclerosis is relapsing-remitting multiple sclerosis.

In a further embodiment, the subject is a human being.

In a further embodiment, the amount is a therapeutically effective amount.

In a further embodiment, the therapeutically effective amount is an amount effective to alleviate a symptom of the form of multiple sclerosis with which the subject is afflicted.

In a further embodiment, the symptom is the frequency of relapses, the frequency of clinical exacerbation, or the accumulation of physical disability.

In a further embodiment, the administration is effected orally, parenterally, rectally or transdermally. In a further embodiment, the administration is effected orally.

In a further embodiment, the method comprises administering R(+)-6-(N-methyl, N-ethyl-carbamoyloxy)-N′-propargyl-1-aminoindan.

In a further embodiment, the method comprises administering a pharmaceutically acceptable salt of R(+)-6-(N-methyl, N-ethyl-carbamoyloxy)-N′-propargyl-1-aminoindan.

In a further embodiment, the pharmaceutically acceptable salt of R(+)-6-(N-methyl, N-ethyl-carbamoyloxy)-N′-propargyl-1-aminoindan is ½ tartrate.

In a further embodiment, the amount of R(+)-6-(N-methyl, N-ethyl-carbamoyloxy)-N′-propargyl-1-aminoindan ½ tartrate is in the range from 0.5 mg to 2000 mg.

In a further embodiment, the salt of R(+)-6-(N-methyl, N-ethyl-carbamoyloxy)-N′-propargyl-1-aminoindan is in crystalline form.

In a further embodiment of the method, the amount of R(+)-6-(N-methyl, N-ethyl-carbamoyloxy)-N′-propargyl-1-aminoindan or the amount of a pharmaceutical salt thereof is an amount that causes at least 60% inhibition of acetylcholinesterase in the blood of the subject.

In a further embodiment of the method, the amount of R(+)-6-(N-methyl, N-ethyl-carbamoyloxy)-N′-propargyl-1-aminoindan or the amount of a pharmaceutical salt thereof is an amount that causes at least 35% inhibition of butyrylcholinesterase in the blood of the subject.

In a further embodiment of the method, the amount of R(+)-6-(N-methyl, N-ethyl-carbamoyloxy)-N′-propargyl-1-aminoindan or the amount of a pharmaceutical salt thereof is an amount that causes at least 55% inhibition of monoamine oxidase A in the subject.

In a further embodiment of the method, the amount of R(+)-6-(N-methyl, N-ethyl-carbamoyloxy)-N′-propargyl-1-aminoindan or the amount of a pharmaceutical salt thereof is an amount that causes at least 81% inhibition of monoamine oxidase B in the subject.

In a further embodiment of the method, the subject is human and the amount of R(+)-6-(N-methyl, N-ethyl-carbamoyloxy)-N′-propargyl-1-aminoindan or the amount of a pharmaceutical salt thereof is 3.3 mg/kg/day-5.0 mg/kg/day.

By 3.3 mg/kg/day-5.0 mg/kg/day it is meant that all hundredth, tenth and integer unit amounts within the range are specifically disclosed as part of the invention. Thus, 3.31, 3.32 . . . 4.99 and 3.4, 3.5 . . . 4.9 mg/kg/day unit amounts are included as embodiments of this invention.

In a further embodiment, the R(+)-6-(N-methyl, N-ethyl-carbamoyloxy)-N′-propargyl-1-aminoindan or the pharmaceutically acceptable salt thereof is in a pharmaceutical composition and at least one pharmaceutically acceptable carrier.

In a further embodiment, in the pharmaceutical composition up to 5% by weight of the pharmaceutical composition is water.

In a further embodiment, the pharmaceutical composition comprises 2-5% water.

In a further embodiment, the pharmaceutical composition comprises 2-3.5% water.

In a further embodiment, in the pharmaceutical composition no more than 0.5% by weight of the pharmaceutical composition is magnesium stearate.

In a further embodiment, the pharmaceutical composition is free of magnesium stearate.

In a further embodiment, in the pharmaceutical composition no more than 1.5% by weight of the pharmaceutical composition is sodium stearyl fumarate.

In a further embodiment, the pharmaceutical composition comprises no more than 0.5% by weight of the composition of sodium stearyl fumarate.

In a further embodiment, the pharmaceutical composition is free of sodium stearyl fumarate.

In a further embodiment, the pharmaceutical composition comprises no more than 0.5% by weight of the composition of stearic acid.

In a further embodiment, in the pharmaceutical composition the crystalline R(+)-6-(N-methyl, N-ethyl-carbamoyloxy)-N′-propargyl-1-aminoindan ½ L-tartrate has a tapped density of at least 0.300 g/ml.

In a further embodiment, the crystalline R(+)-6-(N-methyl, N-ethyl-carbamoyloxy)-N′-propargyl-1-aminoindan ½ L-tartrate has a tapped density of at least 0.400 g/ml.

In a further embodiment, the crystalline R(+)-6-(N-methyl, N-ethyl-carbamoyloxy)-N′-propargyl-1-aminoindan ½ L-tartrate has a tapped density of at least 0.500 g/ml.

In a further embodiment, the crystalline R(+)-6-(N-methyl, N-ethyl-carbamoyloxy)-N′-propargyl-1-aminoindan ½ L-tartrate has a bulk density of at least 0.200 g/ml.

In a further embodiment, the crystalline R(+)-6-(N-methyl, N-ethyl-carbamoyloxy)-N′-propargyl-1-aminoindan ½ L-tartrate has a bulk density of at least 0.250 g/ml.

In a further embodiment, the crystalline R(+)-6-(N-methyl, N-ethyl-carbamoyloxy)-N′-propargyl-1-aminoindan ½ L-tartrate has a tapped density of less than 0.600 g/ml.

In a further embodiment, in the pharmaceutical composition the at least one pharmaceutically acceptable carrier is a first filler, a second filler, a disintegrant, a flow agent, a binder or a lubricant.

In a further embodiment, the lubricant is talc.

In a further embodiment, the talc is present in an amount of up to 4% by weight of the pharmaceutical composition.

In a further embodiment, the lubricant further comprises stearic acid.

In a further embodiment, the stearic acid is present in an amount of up to 2% by weight of the composition.

In a further embodiment, the lubricant is stearic acid.

In a further embodiment, the composition is free of talc.

In a further embodiment, the composition is free of stearic acid.

In a further embodiment, the first filler is mannitol present in an amount of 6 to 16% by weight, the second filler is mannitol granulate present in an amount of 0 to 56% by weight, the disintegrant is starch present in an amount of 15 to 38% by weight, the flow agent is colloidal silicon dioxide present in an amount of 1 to 2% by weight, and the binder is polyvinylpyrolidone present in an amount of 3 to 8% by weight.

In a further embodiment, the first filler is mannitol present in an amount of 6.6% by weight, the second filler is mannitol granulate present in an amount of 56.1% by weight, the disintegrant is starch present in an amount of 15.2% by weight, the flow agent is colloidal silicon dioxide present in an amount of 0.9% by weight, the binder is polyvinylpyrolidone present in an amount of 3.4% by weight, and the lubricant is talc in an amount of 3.8% by weight and stearic acid in an amount of 1.9% by weight.

In a further embodiment, the first filler is mannitol present in an amount of 16.4% by weight, the disintegrant is starch present in an amount of 37.5% by weight, the flow agent is colloidal silicon dioxide present in an amount of 2.1% by weight, the binder is polyvinylpyrolidone present in an amount of 8.4% by weight, and the lubricant is talc in an amount of 3.7% by weight and stearic acid in an amount of 1.9% by weight.

In a further embodiment, the pharmaceutical composition is in the form of tablets, capsules, pills, powders, or granules.

In a further embodiment, the pharmaceutical composition is in tablet form.

The subject invention also provides a method for alleviating a symptom of multiple sclerosis in a subject afflicted with a form of multiple sclerosis comprising administering to the subject an amount of a compound effective to cause at least 60% inhibition of acetylcholinesterase in the blood of the subject thereby alleviating the symptom of multiple sclerosis in the subject. In an embodiment, the compound is R(+)-6-(N-methyl, N-ethyl-carbamoyloxy)-N′-propargyl-1-aminoindan or a pharmaceutically acceptable salt thereof. Such method is applicable to all forms of multiple sclerosis, all symptoms of multiple sclerosis, and all methods of compound administration as described herein.

The subject invention also provides a method for alleviating a symptom of multiple sclerosis in a subject afflicted with a form of multiple sclerosis comprising administering to the subject an amount of a compound effective to cause at least 35% inhibition of butyrylcholinesterase in the blood of the subject thereby alleviating the symptom of multiple sclerosis in the subject. In an embodiment, the compound is R(+)-6-(N-methyl, N-ethyl-carbamoyloxy)-N′-propargyl-1-aminoindan or a pharmaceutically acceptable salt thereof. Such method is applicable to all forms of multiple sclerosis, all symptoms of multiple sclerosis, and all methods of compound administration as described herein.

The subject invention also provides a method for alleviating a symptom of multiple sclerosis in a subject afflicted with a form of multiple sclerosis comprising administering to the subject an amount of a compound effective to cause at least 55% inhibition of monoamine oxidase A in the subject thereby alleviating the symptom of multiple sclerosis in the subject. In an embodiment, the compound is R(+)-6-(N-methyl, N-ethyl-carbamoyloxy)-N′-propargyl-1-aminoindan or a pharmaceutically acceptable salt thereof. Such method is applicable to all forms of multiple sclerosis, all symptoms of multiple sclerosis, and all methods of compound administration as described herein.

The subject invention also provides a method for alleviating a symptom of multiple sclerosis in a subject afflicted with a form of multiple sclerosis comprising administering to the subject an amount of a compound effective to cause at least 81% inhibition of monoamine oxidase B in the subject thereby alleviating the symptom of multiple sclerosis in the subject. In an embodiment, the compound is R(+)-6-(N-methyl, N-ethyl-carbamoyloxy)-N′-propargyl-1-aminoindan or a pharmaceutically acceptable salt thereof. Such method is applicable to all forms of multiple sclerosis, all symptoms of multiple sclerosis, and all methods of compound administration as described herein.

The subject invention also provides use of R(+)-6-(N-methyl, N-ethyl-carbamoyloxy)-N′-propargyl-1-aminoindan or a pharmaceutically acceptable salt thereof in the manufacture of a medicament for the treatment of, or alleviation of symptoms of, a form of multiple sclerosis. Such use has the same embodiments as those specifically disclosed herein in the context of a method.

The subject invention also provides a use of a compound effective to cause at least 60% inhibition of acetylcholinesterase in the blood of the subject in the manufacture of a medicament for alleviating a symptom of multiple sclerosis. Such use has the same embodiments as those specifically disclosed herein in the context of a method. In an embodiment, the compound is R(+)-6-(N-methyl, N-ethyl-carbamoyloxy)-N′-propargyl-1-aminoindan or a pharmaceutically acceptable salt thereof.

The subject invention also provides a use of a compound effective to cause at least 35% inhibition of butyrylcholinesterase in the blood of the subject in the manufacture of a medicament for alleviating a symptom of multiple sclerosis. Such use has the same embodiments as those specifically disclosed herein in the context of a method. In an embodiment, the compound is R(+)-6-(N-methyl, N-ethyl-carbamoyloxy) -N′-propargyl-1-aminoindan or a pharmaceutically acceptable salt thereof.

The subject invention also provides a use of a compound effective to cause at least 55% inhibition of monoamine oxidase A in the blood of the subject in the manufacture of a medicament for alleviating a symptom of multiple sclerosis. Such use has the same embodiments as those specifically disclosed herein in the context of a method. In an embodiment, the compound is R(+)-6-(N-methyl, N-ethyl-carbamoyloxy)-N′-propargyl-1-aminoindan or a pharmaceutically acceptable salt thereof.

The subject invention also provides a use of a compound effective to cause at least 81% inhibition of monoamine oxidase B in the blood of the subject in the manufacture of a medicament for alleviating a symptom of multiple sclerosis. Such use has the same embodiments as those specifically disclosed herein in the context of a method. In an embodiment, the compound is R(+)-6-(N-methyl, N-ethyl-carbamoyloxy)-N′-propargyl-1-aminoindan or a pharmaceutically acceptable salt thereof.

The subject invention also provides a pharmaceutical composition for use in the treatment of, or alleviation of symptoms of, a form of multiple sclerosis, which comprises an amount of R(+)-6-(N-methyl, N-ethyl-carbamoyloxy)-N′-propargyl-1-aminoindan or a pharmaceutically acceptable salt thereof and a pharmaceutically acceptable carrier.

R(+)-6-(N-methyl,N-ethyl-carbamoyloxy)-N′-propargyl-1-aminoindan, also known as (3R)-3-(prop-2-ynylamino)-2,3,-dihydro-1H-inden-5-yl ethylmethylcarbamate, is disclosed in PCT Application Publication No. WO98/27055 (U.S. Pat. No. 6,303,650, issued Oct. 16, 2001 to Chorev), the entire contents of which are incorporated by reference. This compound has been given the nonproprietary name ladostigil.

The present invention thus provides as a compound the R(+)-enantiomer of 6-(N-methyl,N-ethyl-carbamoyloxy)-N′-propargyl-1-aminoindan and pharmaceutically acceptable salts thereof for the treatment of human patients afflicted with multiple sclerosis.

The compound R(+)-6-(N-methyl,N-ethyl-carbamoyloxy)-N′-propargyl-1-aminoindan may be prepared as pharmaceutical compositions particularly useful for the treatment of multiple sclerosis.

Such compositions may comprise the compound of ladostigil or pharmaceutically acceptable salts thereof, together with pharmaceutically acceptable carriers and/or excipients. In the practice of this invention, pharmaceutically acceptable salts include, but are not limited to, the mesylate, maleate, fumarate, tartrate, hydrochloride, hydrobromide, esylate, p-tolunesulfonate, benzoate, acetate, phosphate and sulfate salts.

The compositions may be prepared as medicaments to be administered orally, parenterally, rectally or transdermally. Suitable forms for oral administration include tablets, compressed or coated pills, dragees, sachets, hard or soft gelatin capsules, sublingual tablets, syrups and suspensions; for parenteral administration the invention provides ampoules or vials that include an aqueous or non-aqueous solution or emulsion; for rectal administration there are provided suppositories with hydrophilic or hydrophobic vehicles; and for topical application as ointments and transdermal delivery there are provided suitable delivery systems as known in the art.

As used herein, a “pharmaceutically acceptable” carrier is one that is suitable for use with humans and/or animals without undue adverse side effects (such as toxicity, irritation, and allergic response) commensurate with a reasonable benefit/risk ratio.

Specific examples of pharmaceutical acceptable carriers and excipients that may be used to formulate oral dosage forms of the present invention are described, e.g., in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

A standard method for converting a dosage used in animals to a dosage appropriate for human use is publicly available (Guidance for Industry: Estimating the Maximum Safe Starting Dose in Initial Clinical Trials for Therapeutics in Adult Healthy Volunteers, U.S. Dept. HHS/FDA/CDER (July 2005, at fda.gov/cder/guidance/5541fnl.doc). The dose conversion is species dependent. Interspecies dose conversion consists of dividing the animal dosage by a standard factor in order to derive the dosage for human use. The recommended standard factor for converting to the human equivalent dose for an average 60 kg human based on the animal species is, e.g., 12.3 for mice, 6.2 for rats, 3.1 for cynomolgous monkeys. Alternatively, the human equivalent dose (HED) can be obtained using the following formula:
HED=animal dose in mg/kg*(animal weight in kg/human weight in kg)^{0.33}.

### EXPERIMENTAL DETAILS

### EXAMPLE 1

### Preparation of Ladostigil Tartrate

Ladostigil and specifically ladostigil tartrate can be prepared according to methods disclosed in PCT Application Publication No. WO98/27055.

Alternatively, the crystals of ladostigil tartrate can be improved by preparing them according to the following method:

In a 250 liter reactor, a solution of R(+)-6-(N-methyl, N-ethyl-carbamoyloxy)-N′-propargyl-1-aminoindan (8.3 kg) in isopropanol (52.4 liters) was heated to 60-65° C. The solution was seeded with 50 g of ladostigil tartrate and a solution of L-tartaric acid (2.4 kg) in isopropanol (38.5 liters) was added dropwise over 2.5-3.5 hours. The mixture was maintained at 60-65° C. for 4-15 hr and was then gradually cooled to 0-5° C. over a period of 6 hours. The product was collected in a Guedu FD-2 filter drier and was washed with cold isopropanol (77 liters). The wet material was dried in a filter drier in three stages until moisture content was less than 0.5%. In the first drying stage, the product was dried by static drying for 4 hours at 50-60° C. and under vacuum of less than 50 mbar. In the second drying stage, the product was dried while being stirred for 2 hours at 50-60° C. and under vacuum of less than 50 mbar. In the third drying stage the product was dried while being stirred for 2 hours at 78-82° C. and under vacuum of less than 50 mbar. Two batches of dried ladostigil tartrate (9.67 kg) were obtained. The bulk density/tapped density of the two batches were 0.290/0.535 (g/ml) and 0.245/0.450 (g/ml), respectively.

This procedure may be performed, however, without the seeding step. In addition, the dried material may be milled in a Comil 197 Double screen 018R 6000 rpm in order to improve content uniformity.

Formulations

The formulations of ladostigil tartrate described herein, and specifically that shown in Table 1, have been found to be uniform and stable:

TABLE 1
Formulations of Ladostigil Tartrate

| | Tablet | Tablet | Tablet | |
|---|---|---|---|---|
| | equivalent to | equivalent to | equivalent to | |
| | 20 mg | 50 mg | 80 mg | |
| Excipient | Ladostigil | Ladostigil | Ladostigil | |
| (mg) | Tartrate base | Tartrate base | Tartrate base | Function |
| Ladostigil | 25.6 | 64.0 | 102.4 | Drug |
| Tartrate | | | | Substance |
| Mannitol | 13.4 | 33.5 | 53.6 | Filler |
| USP/BP | | | | |
| Pregelatinized | 32.0 | 80.0 | 128.0 | Disintegrant |
| starch (starch | | | | |
| 1500 NF) | | | | |
| Syloid 244 | 1.8 | 4.5 | 7.2 | Disintegrant |
| (Colloidal | | | | and flowing |
| Silicon | | | | agent |
| Dioxide) | | | | |
| Polividone 30 | 7.2 | 18.0 | 28.8 | Binder |
| (PVP) | | | | |
| Mannitol | 120.0 | — | — | Filler |
| granulate | | | | |
| Stearic acid | 4.0 | 4.0 | 6.4 | Lubricant |
| Talc | 8.0 | 8.0 | 12.8 | Lubricant |
| Isopropyl | q.s. | q.s. | q.s. | |
| alcohol | | | | |
| Total tablet | 212.0 | 212.0 | 339.2 | |
| weight | | | | |

### EXAMPLE 2

### Effect of Ladostigil Tartrate on Treatment of Chronic Progressive EAE

Study Objective

MOG (Myelin oligodendrocyte glycoprotein) is a member of the immunoglobulin superfamily expressed exclusively in CNS myelin. It is one of the most encephalitogenic proteins and is widely used to induce EAE in different rodent strains. In C57BL/6 mice, immunization with the MOG peptide pMOG35-55 in CFA induces chronic progressive EAE. Ladostigil was tested in this model in order to assess its ability to inhibit the disease.

Procedure

EAE was induced by subcutaneous injection of encephalitogenic emulsion at a volume of 0.2 ml/mouse in the right flank. On the day of induction, pertussis toxin was injected intraperitoneally at a volume dose of 0.2 ml/mouse. The injection of the pertussis toxin was repeated after 48 hours. At the end of the experiment, 6-7 brains from control and the ladostigil groups were dissected and frozen at −70° C. for CHE (cholinesterase) determination and 6-7 brains were dissected and frozen for MAO (monoamine oxidase) determination. Before sacrifice, blood for ChE determination was taken on heparine, hemolysed 1/20 in 10 mM cold phosphate buffer pH=6.0 and stored at −70° C. until the assay. The procedure is summarized in Table 2.

TABLE 2

| Day | Test procedure |
|---|---|
| 0 | Subcutaneous injection of MOG into right flank, |
| | intraperitoneal injection of pertussis toxin, |
| | beginning of daily ladostigil treatment. |
| 2 | Intraperitoneal injection of pertussis toxin |
| 10 | Initiation of scoring of mice for EAE clinical signs |
| 49 | Termination of study |

Materials

Reagents

1. MOG, “Novetide” lot #800533-81.

2. Mycobacterium tuberculosis H37 RA (MT) lot #3044883.

3. Pertussis toxin, “Sigma” lot #073K1438.

4. Complete Freund's Adjuvant (CFA), “Sigma” lot #102K8930.

Tested Compounds

1. GA, batch# 242907804.

2. ladostigil, batch# 332600105.

Experimental Animals

90 female C57Bl/6 mice 8-10 weeks old obtained from Harlan animal breeding center were used in the study. Mice were allocated randomly into 6 groups:

TABLE 3

| | | Dose | | | |
|---|---|---|---|---|---|
| # | Group | (mg/kg) | Route | Start | N |
| 1 | Control (DDW) | | gavage | | 15 |
| 2 | GA-blocking | 12.5 | with | On day 0 | 15 |
| | | | inoculum | | |
| 3 | Ladostigil | 10.2 | gavage | On day 0 | 15 |
| 4 | (salt) | 25.5 | gavage | On day 0 | 15 |
| 5 | | 51 | gavage | On day 0 | 15 |
| 6 | | 70.1 | gavage | On day 0 | 15 |

Preparation of Encephalitogenic Emulsion

Oil portion: CFA (containing 1 mg/ml MT) was enriched to the concentration of 5 mg/ml: 64 mg/MT was added to 16 ml CFA.

Liquid portion:

Groups 1, 3-8:

23.25 mg MOG was diluted in 15.5 ml PBS (1.5 mg/ml, 150 μg/0.1 ml/mouse).

Group 2:

10 mg GA was diluted in 4 ml PBS (2.5 mg/ml, 250 μg/0.1 ml/mouse). 6 mg MOG was added to this solution.

The emulsification was made from equal parts of oil and liquid portions (1:1) in two syringes connected to each other with Leur lock, was transferred to insulin syringe and 0.2 ml was injected to the right flank of each mouse.

Preparation of Pertussis Toxin

70 μl pertussis toxin (200 μg/ml) was added to 27.93 ml saline to yield 500 ng/ml (100 ng/0.2 ml/mouse).

Preparation of Ladostigil

The high dose (70.1 mg/10 ml/kg) solution (420.6 mg in 60 ml DDW) was prepared once weekly and stored at −4° C. The dilutions for the other doses were made from the stock solution daily according to Table 4:

TABLE 4

| | | Stock | |
|---|---|---|---|
| Dose | Ratio | solution (ml) | DDW (ml) |
| 51 mg/kg | 1:1.37 | 2.62 | 0.98 |
| 25.5 mg/kg | 1:2.75 | 1.31 | 2.29 |
| 10.2 mg/kg | 1:6.87 | 0.52 | 3.08 |

Both compounds were administered by gavage once daily during the whole experiment (30 days) at a volume of 0.2 ml/mouse.

EAE Clinical Signs

The mice were observed daily from the 10^{th} day post-EAE induction and the EAE clinical signs were scored. The scores were recorded on observation cards according to the grades described in Table 5:

TABLE 5
Evaluation of the EAE clinical signs

| Score | Signs | Description |
|---|---|---|
| 0 | Normal behavior | No neurological signs |
| 1 | Distal limp tail | The distal part of the tail |
| | | is limp and droops |
| 1.5 | Complete limp tail | The whole tail is loose and |
| | | droops |
| 2 | Righting reflex | Animal has difficulties to |
| | | return on its feet when it |
| | | is laid on its back |
| 3 | Ataxia | Wobbly walk - when the |
| | | mouse walks the hind legs |
| | | are unsteady |
| 4 | Early paralysis | The mouse has difficulties |
| | | standing on its hind legs |
| | | but still has remnants of |
| | | movement |
| 5 | Full paralysis | The mouse can't move it's |
| | | legs at all, it looks |
| | | thinner and emaciated |
| 6 | Moribund/death | |

Assessment of EAE Results

Calculation of the Incidence of Disease

The number of sick animals (animals with a score of ≧1 at any point) in each group was summed.

The incidence of disease and the % activity were calculated as follows:

$$Incidence of disease = \frac{No . of sick mice in group}{No . of mice in group} ⨯ 100$$

$$\% activity ( 1 ) = ( 1 - \frac{\% incidence in treated group}{\% incidence in control group} ) ⨯ 100$$

$$( 1 ) = ( \% activity according to incidence )$$

Calculation of the Mean Maximal Score (MMS)

The maximal scores of all mice in each group were summed.

The mean maximal score and the % activity of the group were calculated as follows:

$$Mean Maximal score = \frac{\sum Maximal score of each mouse}{No . of mice in the group}$$

$$\% activity ( 2 ) = ( 1 - \frac{MMS of treated group}{MMS of control group} ) ⨯ 100$$

$$( 2 ) = ( \% activity according to MMS )$$

Calculation of the Group Mean Score (GMS)

The individual scores (IMS) of each of the mice during the observation period were summed (score 6 will be counted forward).

The GMS of the group and its % activity were calculated as follows:

$$Mean score = \frac{\sum IMS of each mouse}{No . of mice in the group}$$

$$\% activity ( 3 ) = ( 1 - \frac{GMS of treated group}{GMS of control group} ) ⨯ 100$$

$$( 3 ) = ( \% activity according to GMS )$$

Note: The GMS score may not be appropriate when some of the animals die during the study.

Calculation of the Mean Time of Onset of Disease
The time of disease onset (days) for each mouse in the group was summed.
The mean onset of disease for the group was calculated.
Mice that did not develop EAE were taken into consideration for calculation of onset of disease. Their dates of onset were calculated as day of termination +1.

Calculation of the Mean Duration of Disease
The disease duration (days) of each mouse in each group was summed.
The mean disease duration of the group was calculated.
The disease duration of mice that did not develop EAE was considered as zero.

Note: The mean duration parameter may not be appropriate when animals die during the study.

Results

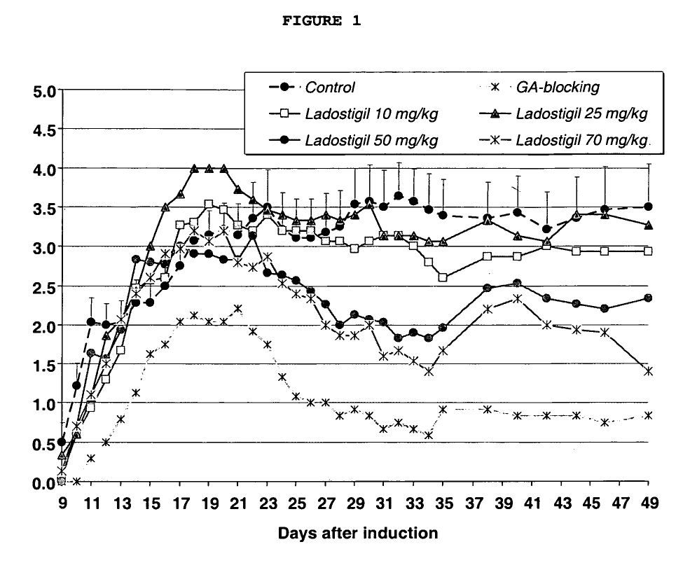

The results are shown in Table 6 and FIG. 1.

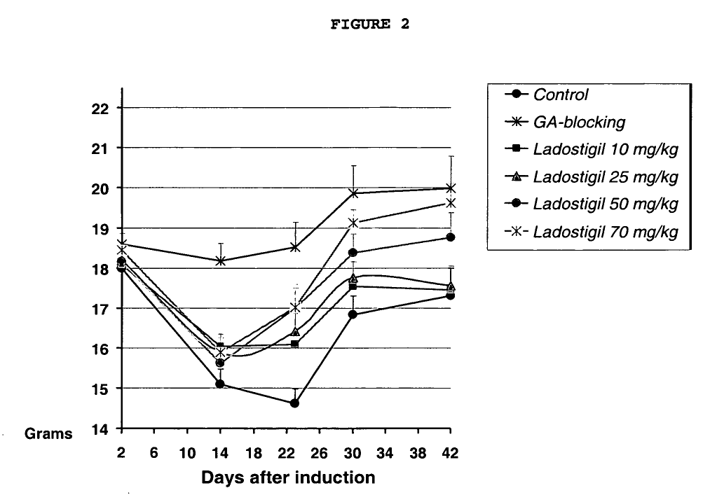

Weight gain results are shown in Table 7 and FIG. 2.

The differences in weight gain were correlated with the disease severity, i.e., stronger disease severity resulted in a greater weight loss. These differences were statistically significant (p<0.000, Manova). According to the subsequent Post Hoc comparison (LSD), all the groups were different from the control (Table 7). The pair-wise comparison, which tests the differences on each certain day when the mice were weighed, revealed that the initial weight of all the groups was equal. On day 14, four days after the onset of the disease, sick mice lost an average of 2 g of weight. The average mean weight of GA-blocked mice was reduced by only 0.4 g. On day 23 the control group reached the lowest weight, 14.6 g. The highest dose of ladostigil was comparable with the positive control on day 30.

TABLE 6
Percentage of inhibition by ladostigil in the EAE mice

| | | Parameter | | | | | | |
|---|---|---|---|---|---|---|---|---|
| Group | | | | Mean | Group | | Mean | Mean |
| | dose, | | | Maximal | Mean | % | Onset, | Disease |
| | mg/kg | Incidence | Mortality | Score | Score | Inhibition | days | Duration, days |
| Control | | 14/14 | 4/14 | 4.39 ± 0.4 | 3.04 ± 1.4 | | 10.7 ± 2.2 | 36.0 ± 10.2 |
| GA | 250 | 14/15 | 0/15 | 3.13** ± 1.0 | 1.38*** ± 0.8 | 54.6% | 13.7** ± 10.2 | 22.9*** ± 13.2 |
| blocking | | | | | | | | |
| Ladostigil | 10.2 | 15/15 | 2/15 | 4.33 ± 1.1 | 2.75 ± 1.3 | 9.5% | 12.0 ± 2.4 | 32.7* ± 9.4 |
| | 25.5 | 15/15 | 3/15 | 4.33 ± 1.0 | 3.08 ± 1.4 | −1.3% | 11.6 ± 2.1 | 35.3 ± 8.8 |
| | 51 | 15/15 | 1/15 | 3.83 ± 1.1 | 2.26 ± 1.1 | 25.7% | 11.4 ± 3.1 | 31.3 ± 13.2 |
| | 70.1 | 15/15 | 1/15 | 3.73 ± 1.0 | 2.03* ± 1.0 | 33.2% | 11.6 ± 2.3 | 29.7* ± 11.2 |

TABLE 7
Weight gain by ladostigil in the EAE mice

| Group | | Parameter | | | | |
|---|---|---|---|---|---|---|
| | dose, | Days After Induction | | | | |
| | mg/kg | 2 | 14 | 23 | 30 | 42 |
| Control | | 18.0 ± 1.3 | 15.1 ± 1.5 | 14.6 ± 1.29 | 16.8 ± 1.6 | 17.3 ± 2.3 |
| GA | 250 | 18.6 ± 0.8 | 18.2*** ± 1.4 | 18.5*** ± 1.9 | 19.9*** ± 1.5 | 20.0** ± 1.5 |
| blocking | | | | | | |
| Ladostigil | 10.2 | 18.1 ± 1.0 | 16.0 ± 1.7 | 16.1* ± 2.3 | 17.6 ± 2.5 | 17.5 ± 2.9 |
| | 25.5 | 18.2 ± 0.8 | 15.9 ± 1.7 | 16.4** ± 1.5 | 17.8 ± 1.4 | 17.6 ± 1.7 |
| | 51 | 18.2 ± 0.9 | 15.6 ± 1.6 | 17.0*** ± 1.9 | 18.4** ± 1.8 | 18.8* ± 2.3 |
| | 70.1 | 18.5 ± 0.8 | 15.9 ± 1.4 | 17.0*** ± 2.3 | 19.1*** ± 1.2 | 19.6** ± 1.8 |

### EXAMPLE 3

### Effect of Ladostigil Tartrate on Treatment of Relapsing Remitting EAE

Procedure

This experiment is conducted to evaluate whether ladostigil tartrate is effective in alleviating EAE. EAE is induced in mice by the administration of myelin oligodendrocyte glycoprotein (MOG). Injection of MOG into 129/SvEv mice produces a relapsing remitting EAE (murine model of relapsing remitting multiple sclerosis).

One week before MOG immunization, 129/SvEv mice receive a sub-optimal dose of ladostigil tartrate or saline vehicle. Over a period of 1-2 months following MOG immunization, clinical scores are monitored by weighing animals on a daily basis and grading the severity of EAE. In addition to clinical evaluation, groups of mice are sacrificed at weekly intervals for routine histological examination (hematoxylin-eosin and luxol fast blue for evidence of inflammation and demyelination, respectively). Also, cellular infiltrates are examined for CD3 (T lymphocytes), CD56 (natural killer cells) and CD19 (B cell immunohistochemistry). Loss of neurons and oligodendrocytes is assessed using immunohistochemistry for NeuN and glutathione-S-transferase pi isoform, respectively. Furthermore, axonal integrity is examined using immunohistochemistry for β-amyloid precursor protein or neurofilament, and by Bielchowsky silver stains. The neuropathological assessments are focused on the optic nerve, lumbar/sacral cord and brain stem.

Results

In comparison to the mice receiving saline vehicle, the mice receiving treatment with ladostigil tartrate exhibit a lower clinical score and a smaller area under the curve. The results demonstrate that ladostigil tartrate decreases both the incidence and severity of EAE, as compared to control.

### EXAMPLE 4

### Cholinesterase (ChE) Assay

From the mice of Example 2, acetylcholinesterase (AChE) and butyrylcholinesterase (BChE) activities were determined by a modification of Ellman's spectophotometric method using acetyltiocholine (ATC) and butyryltiocholine (BTC) as substrates.

ChE in Brains

Homogenate of 100 mg brain tissue in 1 ml 100 mM phosphate buffer pH=8.0 was prepared. 0.4 ml of crude homogenate was added to 1.2 ml pre-warmed 4 mM 5,5′-Dithiobis(2-nitrobenzoic acid) (DTNB or Ellman's reagant); 30 μl from this mixture were added to 210 μl pre-warmed (28° C.) 50 mM phosphate buffer in a microplate; and the reaction was started by addition of 10 μl ATC or BTC (final concentration=1 mM) or water (for the blank reaction) The increase in optical density (OD) at 412 nm was monitored for 3 minutes and the slope of the straight line obtained by linear regression provided the activity in terms of ΔOD/min using SOFTMAX® software. Blank reaction was subtracted.

Conversion of ΔOD/min into Enzyme Activity was Performed as Follows:
Unit enzyme/gram tissue=OD/min*0.25/10.54*4*1000/30*11

10.54=absorbance coefficient of DTNB per well (per 1 ml)=molar absorbance

(13,600)*well height (0.775 cm)/1000

0.25=reaction volume

11=homogenate dilution weight/volume

ChE in Blood

The same procedure was performed as with brain homogenate using 50 μl of ¼ hemolysate/DTNB mixture.

Enzyme activity in the blood was calculated in units/1 ml whole blood.

BuChE activity was calculated directly from the ΔOD/min of BTC.

AChE activity was calculated as follows:
AChE activity=ΔOD/min (determined with ATC)−ΔOD/min (determined with BTC)/0.7

0.7=BChE activity on BTC/BChE activity on ATC. It was determined by using 1.25-10⁻⁶M BW281C51 to inhibit AChE and with ATC and BTC as substrates.

Results

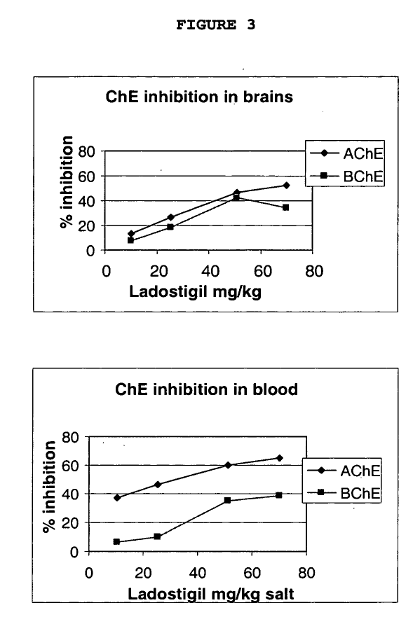

The results are shown in Tables 8-9 and FIG. 3.

The ChE activity observed in brains was mostly acetylcholinesterase. Dose dependent inhibition was observed with both enzymes in brain and blood up to 51 mg/kg/day (salt) with only small further increase or no increase of % inhibition at 70.1 mg/kg/day. Ladostigil doses that were effective in ameliorating EAE (51 and 70.1 mg/kg/day) exerted 47-52% inhibition of AChE in brain and 60-65% inhibition of AChE in blood. BChE was inhibited to an extent of 35-42%.

TABLE 8
Percentage of ChE inhibition by ladostigil in the
brains of EAE mice

| | | Brain | | | Brain |
|---|---|---|---|---|---|
| | Dose | AChE U/g | Brain AchE % | Brain | BchE % |
| | mg/kg salt | (Total ChE) | inhibition | BuChE U/g** | inhibition |
| Control | | 10.13 +/− 1.16 | 0 | 0.38 +/− 0.05 | 0 |
| Ladostigil | 10.2 | 8.77 +/− 1.23 | 13.4 | 0.35 +/− 0.04 | 7.9 |
| Ladostigil | 25.5 | 7.42 +/− 1.29 | 26.8* | 0.31 +/− 0.04 | 18.4 |
| Ladostigil | 51 | 5.41 +/− 0.6 | 46.6* | 0.22 +/− 0.03 | 42.1* |
| Ladostigil | 70.1 | 4.85 +/− 0.65 | 52.2* | 0.25 +/− 0.06 | 34.2* |

*P < 0.01 One-way Anova + Dunnett test
**Results are on the limit of assay sensitivity

TABLE 9
Percentage of ChE inhibition by ladostigil in the
blood of EAE mice

| | | Blood | | Blood | Blood |
|---|---|---|---|---|---|
| | Dose mg/kg | AChE | Blood AchE % | BuChE | BchE % |
| | salt | U/ml | inhibition | U/ml | inhibition |
| Control | | 1.36 +/− 0.18 | 0 | 1.63 +/− 0.2 | 0 |
| Ladostigil | 10.2 | 0.85 +/− 0.17 | 37.4* | 1.52 +/− 0.24 | 6.7 |
| Ladostigil | 25.5 | 0.73 +/− 0.12 | 46.3* | 1.47 +/− 0.18 | 10.1 |
| Ladostigil | 51 | 0.54 +/− 0.1 | 60.2* | 1.06 +/− 0.21 | 35.3* |
| Ladostigil | 70.1 | 0.48 +/− 0.1 | 64.9* | 1.01 +/− 0.18 | 38.4* |

*P < 0.01 One-way Anova + Dunnett test

### EXAMPLE 5

### Monoamine Oxidase (MAO) Assay

From the mice of Example 2, the level of MAO (MAO-A and MAO-B) inhibition was determined using ¹⁴C-Labeled 2-phenylethylamine and 5-hydroxytryptamine as substrates.

The MAO enzyme obtained from a homogenate of rat brain in 0.3M sucrose, which was centrifuged at 600 g for 15 minutes. The supernatant is diluted appropriately in 0.05M phosphate buffer, and pre-incubated with serial dilutions of test compounds for 20 minutes at 37° C. ¹⁴C-Labeled substrates (2-phenylethylamine, hereinafter PEA; 5-hydroxytryptamine, hereinafter 5-HT) are then added, and the incubation continued for a further 20 minutes (PEA), or 30-45 minutes (5-HT). Substrate concentrations used are 50 μM (PEA) and 1 mM (5-HT). In the case of PEA, enzyme concentration is chosen so that not more than 10% of the substrate is metabolized during the course of the reaction. Deaminated products are extracted into toluene-ethyl acetate (1:1 v/v) containing 0.6% (w/v) 2,5-diphenyloxazole (ppo) prior to determination by liquid scintillation counting. Radioactivity in the eluate indicates the production of neutral and acidic metabolites formed as a result of MAO activity. Activity of MAO in the sample is expressed as a percentage of control activity in the absence of inhibitors after subtraction of appropriate blank values. The activity determined using PEA as substrate is referred to as MAO-B, and that determined using 5-HT as MAO-A. Concentrations of inhibitor producing 50% inhibition of substrate metabolism (IC₅₀) are calculated from the inhibition curves.

Results

The results are shown in Table 10.

Dose-dependent MAO-A and MAO-B inhibition was observed, with MAO-B inhibition being higher than MAO-A inhibition. The doses of 51 and 70.1 mg/kg/day that were effective in ameliorating EAE disease caused 81-89% inhibition of MAO-B and 55-60% MAO-A inhibition.

TABLE 10
MAO activity and % MAO inhibition by ladostigil
in the brains of EAE mice

| | | | MAO-A | Brain | MAO-B | Brain |
|---|---|---|---|---|---|---|
| | | Dose | activity | MAO-A % | activity | MAO-B % |
| Group | N | mg/kg | dpm | inhibition | dpm | inhibition |
| Control | 7 | | 4052 +/− 652 | 0 | 12855 +/− 2365 | 0 |
| Ladostigil | 6 | 10.2 | 3702 +/− 119 | 9 | 8784 +/− 1047 | 32* |
| Ladostigil | 6 | 25.5 | 2672 +/− 128 | 34* | 4258 +/− 467 | 67* |
| Ladostigil | 6 | 51 | 1822 +/− 115 | 55* | 2424 +/− 1122 | 81* |
| Ladostigil | 7 | 70.1 | 1621 +/− 158 | 60* | 1382 +/− 250 | 89* |

P < 0.01 One-way Anova + Dunnett test

1. A method of treating a subject afflicted with a form of multiple sclerosis comprising administering to the subject an amount of R(+)-6-(N-methyl, N-ethyl-carbamoyloxy)-N′-propargyl-1-aminoindan or a pharmaceutically acceptable salt thereof.

2. The method of claim 1, wherein the form of multiple sclerosis is relapsing-remitting multiple sclerosis.

3. The method of claim 1, wherein the subject is a human being.

4. The method of claim 1, wherein the amount is a therapeutically effective amount.

5. The method of claim 4, wherein the therapeutically effective amount is an amount effective to alleviate a symptom of the form of multiple sclerosis with which the subject is afflicted.

6. The method of claim 5, wherein the symptom is the frequency of relapses, the frequency of clinical exacerbation, or the accumulation of physical disability.

7. The method of claims 1, wherein the administration is effected orally, parenterally, rectally or transdermally.

8. The method of claim 7, wherein the administration is effected orally.

9. (canceled)

10. The method of claim 1, wherein the method comprises administering a pharmaceutically acceptable salt of R(+)-6-(N-methyl, N-ethyl-carbamoyloxy)-N′-propargyl-1-aminoindan.

11. The method of claim 10, wherein the pharmaceutically acceptable salt of R(+)-6-(N-methyl, N-ethyl-carbamoyloxy)-N′-propargyl-1-aminoindan is ½ tartrate.

12. The method of claim 11, wherein the amount of R(+)-6-(N-methyl, N-ethyl-carbamoyloxy)-N′-propargyl-1-aminoindan ½ tartrate is in the range from 0.5 mg to 2000 mg.

13. The method of claim 10, wherein the salt of R(+)-6-(N-methyl, N-ethyl-carbamoyloxy)-N′-propargyl-1-aminoindan is in crystalline form.

14. The method of claim 1, wherein the amount of R(+)-6-(N-methyl, N-ethyl-carbamoyloxy)-N′-propargyl-1-aminoindan or the amount of a pharmaceutical salt thereof is an amount that causes at least 60% inhibition of acetylcholinesterase in the blood of the subject.

15. The method of claim 1, wherein the amount of R(+)-6-(N-methyl, N-ethyl-carbamoyloxy)-N′-propargyl-1-aminoindan or the amount of a pharmaceutical salt thereof is an amount that causes at least 35% inhibition of butyrylcholinesterase in the blood of the subject.

16. The method of claim 1, wherein the amount of R(+)-6-(N-methyl, N-ethyl-carbamoyloxy)-N′-propargyl-1-aminoindan or the amount of a pharmaceutical salt thereof is an amount that causes at least 55% inhibition of monoamine oxidase A in the subject.

17. The method of claim 1, wherein the amount of R(+)-6-(N-methyl, N-ethyl-carbamoyloxy)-N′-propargyl-1-aminoindan or the amount of a pharmaceutical salt thereof is an amount that causes at least 81% inhibition of monoamine oxidase B in the subject.

18. The method of claim 1, wherein the subject is human and the amount of R(+)-6-(N-methyl, N-ethyl-carbamoyloxy)-N′-propargyl-1-aminoindan or the amount of a pharmaceutical salt thereof is 3.3 mg/kg/day-5.0 mg/kg/day.

19. The method of claim 1, wherein the R(+)-6-(N-methyl, N-ethyl-carbamoyloxy)-N′-propargyl-1-aminoindan or the pharmaceutically acceptable salt thereof is in a pharmaceutical composition which also comprises at least one pharmaceutically acceptable carrier.

20. The method of claim 19, wherein in the pharmaceutical composition up to 5% by weight of the pharmaceutical composition is water.

21. The method of claim 19, wherein in the pharmaceutical composition no more than 0.5% by weight of the pharmaceutical composition is magnesium stearate.

22. The method of claim 19, wherein in the pharmaceutical composition no more than 1.5% by weight of the pharmaceutical composition is sodium stearyl fumarate.

23. The method of claim 19, wherein in the pharmaceutical composition the crystalline R(+)-6-(N-methyl, N-ethyl-carbamoyloxy)-N′-propargyl-1-aminoindan ½ L-tartrate has a tapped density of at least 0.300 g/ml.

24. The method of claim 19, wherein in the pharmaceutical composition the at least one pharmaceutically acceptable carrier is a first filler, a second filler, a disintegrant, a flow agent, a binder or a lubricant.

25. The method of claim 24, wherein the first filler is mannitol present in an amount of 6 to 16% by weight, the second filler is mannitol granulate present in an amount of 0 to 56% by weight, the disintegrant is starch present in an amount of 15 to 38% by weight, the flow agent is colloidal silicon dioxide present in an amount of 1 to 2% by weight, and the binder is polyvinylpyrolidone present in an amount of 3 to 8% by weight.

26. The method of claim 19, wherein the pharmaceutical composition is in the form of tablets, capsules, pills, powders, or granules.

27. A method for alleviating a symptom of multiple sclerosis in a subject afflicted with a form of multiple sclerosis comprising administering to the subject an amount of a compound effective to cause at least 60% inhibition of acetylcholinesterase in the blood of the subject thereby alleviating the symptom of multiple sclerosis in the subject.

28. A method for alleviating a symptom of multiple sclerosis in a subject afflicted with a form of multiple sclerosis comprising administering to the subject an amount of a compound effective to cause at least 35% inhibition of butyrylcholinesterase in the blood of the subject thereby alleviating the symptom of multiple sclerosis in the subject.

29-36. (canceled)

37. A method for alleviating a symptom of multiple sclerosis in a subject afflicted with a form of multiple sclerosis comprising administering to the subject an amount of a compound effective to cause either:
i. at least 55% inhibition of monoamine oxidase A; or
ii. at least 81% inhibition of monoamine oxidase B, in the subject thereby alleviating the symptom of multiple sclerosis in the subject.
